(19) United States
(12) Patent Application Publication
(10) Pub. No.: US 2014/0128282 A1
(43) Pub. Date: May 8, 2014

(54) IMMUNE FUNCTION BIOMARKERS

(71) Applicants: Rondo P. Middleton, Creve Coeur, MO (US); Ziad S. Ramadan, Manchester, MO (US); Serge Andre Rezzi, Semsales (CH); Sebastiano Collino, Lausanne (CH); Francois Piere Martin, Vuisternens-devant-Romont (CH)

(72) Inventors: Rondo P. Middleton, Creve Coeur, MO (US); Ziad S. Ramadan, Manchester, MO (US); Serge Andre Rezzi, Semsales (CH); Sebastiano Collino, Lausanne (CH); Francois Piere Martin, Vuisternens-devant-Romont (CH)

(73) Assignee: NESTEC SA, Vevey (CH)

(21) Appl. No.: 14/074,422

(22) Filed: Nov. 7, 2013

Related U.S. Application Data
(60) Provisional application No. 61/723,867, filed on Nov. 8, 2012.

(51) Int. Cl. C12Q 1/68

(52) U.S. Cl. CPC C12Q 1/6883
USPC 506/9; 506/16

(57) ABSTRACT

The invention provides biomarkers associated with age related immune function and the use of the biomarkers to identify compositions useful for strengthening immune function in animals and to determine if an animal is responding to treatment targeted to strengthen the immune system.

### CROSS REFERENCE TO RELATED APPLICATIONS

This application claims priority to U.S. Provisional Application No. 61/723,867 filed Nov. 8, 2012, the disclosure of which is incorporated herein by this reference.

### BACKGROUND OF THE INVENTION

1. Field of the Invention

The invention relates generally to the field of nutritional support of health and immunity in animals. In particular, the invention provides biomarkers associated with immune function, particularly biomarkers associated with age related changes in immune function, the use of the biomarkers to identify compositions useful for strengthening immune function in animals, and to determine if an animal is responding to treatment targeted to strengthen the immune system.

2. Description of Related Art

The gradual decline in immune system function that accompanies aging is known as immune senescence. This decline involves both an animal's capacity to respond to infections and the development of long term immunity. In addition to infectious diseases, an older animal is also more susceptible to other clinical conditions such as cancer, cardiovascular disease, neurological disorders and chronic inflammatory disorders. The identification of biomarkers associated with aging can be used to characterize an animal's immune system functionality. It can also be used to detect agents useful for strengthening immune system function and to monitor the effectiveness of treatment.

Biomarkers associated with immune function are known. However, the known biomarkers are mostly pro-inflammatory proteins or pathogen specific gene expression. US 2007/0150202 to Weigand et al. describe the use of c-reactive proteins and cytokines such as interleukin-6 (IL-6) to assess pro-inflammatory immune health of an individual. US 2004/0038201 to Nau et al. describe stimulus specific gene expression profiles to detect infection by a pathogen. US 2005/0002862 to Alters et al. describe biological markers for evaluating therapeutic treatment of inflammation and autoimmune disorders.

Despite the availability of the approaches summarized above, there remains a need for biomarkers associated with age related immune function and for methods to screen for agents that can strengthen immune function. The present invention satisfies this need.

### SUMMARY OF THE INVENTION

It is, therefore, an object of the present invention to provide a combination comprising a plurality of biomarkers associated with immune function that are differentially expressed in samples from old animals compared with samples from young animals.

It is a further object of the invention to provide methods for determining if a composition is effective in strengthening the immune function in an animal.

It is another object of the invention to provide methods for determining if an animal is responding to treatment with a composition suitable for strengthening immune function.

One or more of these other objects are achieved using novel collections of biomarkers associated with immune function that are differentially expressed in samples from old animals compared with samples from young animals.

Other and further objects, features, and advantages of the invention will be readily apparent to those skilled in the art.

### DETAILED DESCRIPTION OF THE INVENTION

### Definitions

As used throughout, ranges are used herein as shorthand, so as to avoid having to set out at length and describe each and every value within the range. Any appropriate value within the range can be selected, where appropriate, as the upper value, lower value, or the terminus of the range. It is understood that any and all whole or partial integers between any ranges or intervals set forth herein are included herein.

As used herein and in the appended claims, the singular form of a word includes the plural, and vice versa, unless the context clearly dictates otherwise. Thus, the references “a,” “an,” and “the” are generally inclusive of the plurals of the respective terms. For example, reference to “an animal”, “a method”, or “a substance” includes a plurality of such “animals”, “methods”, or “substances”. Similarly, the words “comprise”, “comprises”, and “comprising” are to be interpreted inclusively rather than exclusively.

The term “animal” means a human or other animal, including avian, bovine, canine, equine, feline, hicrine, murine, ovine, and porcine animals. When the term is used in the context of comparing test subjects, the animals that are compared are animals of the same species and possibly of the same race or breed. A “companion animal” is any domesticated animal, and includes, without limitation, cats, dogs, rabbits, guinea pigs, ferrets, hamsters, mice, gerbils, horses, cows, goats, sheep, donkeys, pigs, and the like. Preferably, the animal is a human or a companion animal such as a canine or feline.

The term “differential expression” or “differentially expressed” means increased or unregulated gene expression or means decreased or downregulated gene expression as detected by the absence, presence, or change in the amount of transcribed messenger RNA or translated protein in a sample, or means an increase or decrease in the amount of protein present in a sample.

The term “sample” means any animal tissue or fluid containing, e.g., polynucleotides, polypeptides, antibodies, metabolites, and the like, including cells and other tissue containing DNA and RNA. Examples include adipose, blood, cartilage, connective, epithelial, lymphoid, muscle, nervous, sputum, and the like. A sample may be solid or liquid and may be DNA, RNA, cDNA, bodily fluids such as blood or urine, cells, cell preparations or soluble fractions or media aliquots thereof, chromosomes, organelles, and the like.

“Young” refers generally to an individual in young adulthood, i.e., matured past puberty or adolescence, as would be defined by species, or by strain, breed or ethnic group within a species, in accordance with known parameters. Typically a young feline is less than seven years of age.

“Aged” or “old,” as used herein, refers to an individual who is physically or chronologically within the last 30% of its average life expectancy, as determined by species, or by strain, breed or ethnic group within a species, in accordance with known parameters. Typically an old feline is greater than eleven years.

“Middle-aged” refers generally to an individual that is in between young and old. Typically a middle-aged feline is seven to eleven years of age.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to and does not limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the invention, the preferred compositions, methods, articles of manufacture, or other means or materials are described herein.

All patents, patent applications, publications, and other references cited or referred to herein are incorporated herein by reference to the extent allowed by controlling law. The discussion of those references is intended merely to summarize the assertions made therein. No admission is made that any such patents, patent applications, publications or references, or any portion thereof, is relevant, material, or prior art. The right to challenge the accuracy and pertinence of any assertion of such patents, patent applications, publications, and other references as relevant, material, or prior art is specifically reserved.

### The Invention

In one aspect, the invention provides a combination comprising a plurality of biomarkers associated with immune function that are differentially expressed in samples from old animals compared with samples from young animals.

In another aspect, the invention provides a combination comprising a plurality of biomarkers associated with immune function that are differentially expressed in samples from middle-aged animals compared with samples from young animals.

In another aspect, the invention provides a combination comprising a plurality of biomarkers associated with immune function that are differentially expressed in samples from old animals compared with samples from middle-aged animals.

In another aspect, the invention provides a method for determining if a composition is effective in strengthening the immune function in an animal comprising: (a) obtaining a baseline sample from the animal prior to administration of the composition; (b) analyzing the baseline sample for one or more biomarkers associated with immune function; (c) administering the composition to the animal for a suitable amount of time; (d) obtaining a treatment sample from the animal after completion of the suitable amount of time; (e) analyzing the treatment sample for one or more biomarkers associated with immune function; and (f) determining if the composition is effective if one or more biomarkers present in the baseline sample is differentially expressed in the treatment sample.

In another aspect, the invention provides methods for determining if an animal is responding to treatment with a composition suitable for strengthening immune function comprising: (a) obtaining a baseline sample from the animal prior to administration of the composition; (b) analyzing the baseline sample for one or more biomarkers associated with immune function; (c) administering the composition to the animal for a suitable amount of time; (d) obtaining a treatment sample from the animal after completion of the suitable amount of time; (e) analyzing the treatment sample for one or more biomarkers associated with immune function; and (f) determining if the animal is responding to treatment if one or more biomarker present in the baseline sample is differentially expressed in the treatment sample.

The inventions are based upon the discovery of biomarkers in immune cells that were differentially expressed in samples from old animals and middle-age animals compared to samples from young animals. The markers identified can be used to monitor the effectiveness of therapies targeted at improving the animals' immune function.

The biomarkers of the present invention were identified using multiple technologies including leukocyte gene expression changes, changes in cytokines, chemokines and adipokine proteins and immune cell population changes. In various embodiments, the biomarkers associated with immune function include proteins and genes.

In some embodiments, the biomarker associated with immune function is a gene expression marker that is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, HIST1H4I, SYNGR2, PGD, CHMP2A, FLNA, FKBP8, PITPNC1, ABHD5, AGRN, G3BP1, TKT, MAP3K3, CDC37, PDIA6, LOC100128934, SH3BP1, TREX1, GON4L, MAPK13, PDLIM7, LRRC33, VWF, DIAPH1, SLC25A29, C5AR1, WIF1, LY6E, ILK, and RB1. In a preferred embodiment, the biomarker associated with immune function is a gene expression marker that is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, HIST1H4I, SLC25A29, C5AR1, FKBP8, PITPNC1, ABHD5, WIF1, CHMP2A, PDIA6, ILK, and RB 1. In a more preferred embodiment, the biomarker associated with immune function is a gene expression marker that is one or more of NHP2L1, TRIM50, GGH, FAM32A, SIRT6, PDIA6, ATP6AP1, ZC3H7A, ACTL6B, DNAJA2, CFL1, CYB5R3, FKBP15, PHC2, and WTAP.

In some embodiments, the biomarkers associated with immune function are differentially expressed in samples from old animals compared with samples from young animals. In one embodiment the biomarker associated with immune function is a gene expression marker that is one or more of WTAP, NHP2L1, TRIM50, GGH, FAM32A, NUCB1, KIAA1310, ELK1, SLC25A29, C5AR1, FKBP8, ST6GALNAC4, CCT7, PITPNC1, ABHD5, WIF1, ALOX5, CHMP2A, CDC37, and LY6E. In a preferred embodiment, the gene expression marker is one or more of WTAP, NHP2L1, TRIM50, GGH, FAM32A, NUCB1, KIAA1310, ELK1, SLC25A29, C5AR1, FKBP8, ST6GALNAC4, CCT7, PITPNC1, ABHD5, WIF1, ALOX5, and CHMP2A. In a more preferred embodiment, the gene expression marker is one or more of WTAP, NHP2L1, TRIM50, GGH, and FAM32A.

In some embodiments, the biomarkers associated with immune function are differentially expressed in samples from middle-aged animals compared with samples from young animals. In one embodiment, wherein the biomarker associated with immune function is a gene expression marker that is one or more of SIRT6, PDIA6, SPOCK2, ILK, RB1, CFL1, SLC25A29, C5AR1, SH3BP1, LRRC33, and LY6E. In a preferred embodiment, the gene expression marker is one or more of SIRT6, PDIA6, SPOCK2, ILK, RB1, and CFL1. In a more preferred embodiment, the gene expression marker is one or more of SIRT6 and PDIA6.

In some embodiments, the biomarkers associated with immune function are differentially expressed in samples from old animals compared with samples from middle-aged animals. In one embodiment, the biomarker associated with immune function is a gene expression marker that is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, HIST1H4I, SYNGR2, PGD, CHMP2A, FLNA, FKBP8, PITPNC1, ABHD5, AGRN, G3BP1, TKT, MAP3K3, CDC37, PDIA6, LOC100128934, SH3BP1, TREX1, GON4L, MAPK13, PDLIM7, LRRC33, VWF, and DIAPH1. In a preferred embodiment, the gene expression marker is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, and HIST1H4I. In a more preferred embodiment, the gene expression marker is one or more of ATP6AP1, ZC3H7A, ACTL6B, DNAJA2, CFL1, CYB5R3, FKBP15, PHC2, and WTAP.

In some embodiments, the biomarker associated with immune function is a protein that is one or more of Leptin, MCP-1, IFN, IL-12p40, SCF, IL-18, SDF-1, GLP-1, IL-4, PDGF-BB, Insulin, Amylin, Flt-3L, IL-1b, IL-2, IL-6, and IL-10. In a preferred embodiment, the protein is one or more of Leptin and MCP-1.

In some embodiments, the biomarkers associated with immune function are differentially expressed in samples from old animals compared with samples from young animals. In one embodiment, the biomarker associated with immune function is a protein that is one or more of Leptin, MCP-1 (monocyte chemotactic protein-1), IFN (Interferon), IL-12p40 (Interleukin-12 subunit beta), SCF (stem cell factor), IL-18 (Interleukin-18), SDF-1 (stromal cell-derived factor-1), GLP-1 (Glucagon-like peptide-1), IL-4 (Interleukin-4), PDGF-BB (Platelet-derived growth factor), Insulin, Amylin, Flt-3L (Fms-like Tyrosine Kinase-3), IL-1b (Interleukin-1 beta), IL-2 (Interleukin-2), IL-6 (Interleukin-6), and IL-10 (Interleukin-10). In a preferred embodiment, the protein is one or more of Leptin, MCP-1, IFN, IL-12p40, SCF, IL-18, SDF-1, GLP-1, and IL-4. In a more preferred embodiment, the protein is one or more of Leptin and MCP-1.

In some embodiments, the biomarkers associated with immune function that are differentially expressed in samples from old animals compared with samples from middle-aged animals. In one embodiment, the biomarker associated with immune function is a protein that. is one or more of Leptin, SDF-1, SCF, IFN, IL-12p40, IL-4, Insulin, MCP-1, GLP-1, IL-18, PDGF-BB, Amylin, Flt-3L, IL-1b, IL-2, IL-6, and IL-10, preferably Leptin, SDF-1, SCF, and IFN, more preferably Leptin.

In some embodiments, the biomarkers associated with immune function that are differentially expressed in samples from middle-aged animals compared with samples from young animals. In one embodiment, the biomarker associated with immune function is a protein that is one or more of IL-18, MCP-1, Insulin, IL-12p40, GLP-1, IFN, IL-1b, SCF, Flt-3L, IL-6, IL-4, SDF-1, IL-10, IL-2, Leptin, PDGF-BB, and Amylin, preferably IL-18, MCP-1, Insulin, IL-12p40, GLP-1, IFN, and IL-1b, more preferably IL-18 and MCP-1.

Any sample that is of biological origin may be useful in the present invention. Examples include, but are not limited to, blood (serum/plasma), cerebral spinal fluid (CSF), urine, stool, breath, saliva, or biopsy of any tissue. In one embodiment, the sample is a blood sample. In another embodiment, the sample is a red blood sample. In yet another embodiment, the sample is a white blood sample. In one embodiment, the sample is a tissue sample.

In various embodiments, the animal is a human or companion animal. Preferably, the companion animal is a feline such as a cat.

The suitable amount of time for administering a composition suitable for strengthening immune function is any amount of time required to achieve a strengthened immune function. In one embodiment, the suitable amount of time is at least 4 weeks, preferably at least 2 months, more preferably at least 6 months.

In some embodiments, the method for determining if a composition is effective in strengthening the immune function in an animal is determined if one or more biomarkers present in the baseline sample is differentially expressed in the treatment sample. In one embodiment, the determination is based on if two or more biomarkers present in the baseline sample are differentially expressed in the treatment sample. In another embodiment, the determination is based on if three or more biomarkers present in the baseline sample are differentially expressed in the treatment sample.

In some embodiments, the method for determining if a composition is effective in strengthening the immune function in an animal is determined if the amount of biomarker present in the baseline sample is greater compared to the amount present in the treatment sample.

In some embodiments, the method for determining if a composition is effective in strengthening the immune function in an animal is determined if the amount of biomarker present in the baseline sample is less than compared to the amount present in the treatment sample.

In some embodiments, the method for determining if an animal is responding to treatment with a composition suitable for strengthening immune function is determined if one or more biomarker present in the baseline sample is differentially expressed in the treatment sample. In one embodiment, the determination is based on if two or more biomarkers present in the baseline sample are differentially expressed in the treatment sample. In another embodiment, the determination is based on if three or more biomarkers present in the baseline sample are differentially expressed in the treatment sample

In some embodiments, the method for determining if an animal is responding to treatment with a composition suitable for strengthening immune function is determined if the amount of biomarker present in the baseline sample is greater compared to the amount present in the treatment sample.

In some embodiments, the method for determining if an animal is responding to treatment with a composition suitable for strengthening immune function is determined if the amount of biomarker present in the baseline sample is less than compared to the amount present in the treatment sample.

In various embodiments of the invention, changes in gene expression may be measured in one or both of two ways: (1) measuring transcription through detection of mRNA produced by a particular gene; and (2) measuring translation through detection of protein produced by a particular transcript.

Decreased or increased expression can be measured at the RNA level using any of the methods well known in the art for the quantitation of polynucleotides, such as, for example, PCR (including, without limitation, RT-PCR and qPCR), RNase protection, Northern blotting, microarray, macroarray, and other hybridization methods. The genes that are assayed or interrogated according to the invention are typically in the form of mRNA or reverse transcribed mRNA. The genes may be cloned and/or amplified. The cloning itself does not appear to bias the representation of genes within a population. However, it may be preferable to use polyA+ RNA as a source, as it can be used with fewer processing steps.

Decreased or increased expression can be measured at the protein level using any of the methods well known in the art for protein quantitation, such as, for example, western blotting, ELISA, mass spectrometry, etc.

### EXAMPLES

Various aspects of the invention can be further illustrated by the following examples. It will be understood that these examples are provided merely for purposes of illustration and do not limit the scope of the invention disclosed herein unless otherwise specifically indicated.

### Example 1

Thirty-six (36) animals were used for a feline trial. This consisted of an n=12 for each of 3 age groups. Felines (years): less than 7, 7-11 and greater than 11. Animals were all spayed or neutered. Any animal with an infection, disease, fever, recently immunized or has been given medication within 10 days was not be used. Blood collections were drawn in same 5-day workweek on animals fasted overnight. 1.5-2 mL of blood in 3 mL ACD tubes and 6-8 mL of blood in lithium-heparin tubes was collected for felines. A small aliquot from the lithium heparin tubes (prior to WBC/RNA isolation/plasma collection) was used for blood differential staining. The 1.5-2 mLs in the ACD tube were placed in a 4° C. refrigeration pack and shipped overnight or same day for flow cytometry analysis. All remaining samples were processed according to Ambion® RiboPure™-Blood (Life Technologies, Grand Island, N.Y.) protocol except the plasma (separated from the WBC/red blood cells in the Ambion® protocol) was stored at −80° C.

Cell Population Analysis. Peripheral blood smear/differential stain was performed by drawing up blood into a plain capillary tube and placing of small drop of blood on one end of a microscope slide. A second slide was used to by touching the blood drop at a 45 degree angle and pushing the blood across the first slide making a mono-layered feathered edge smear. Blood was allowed to dry completely and stained with Wright Stain. One drop of immersion oil was placed in the middle of the blood smear and viewed on an Olympus® BX51 microscope (Shinjuku, Japan) at 100× magnification. Percentage of monocytes, lymphocytes, bands, mature neutrophils, eosinophils and basophils were determined.

A resistant z-score rule was applied to the outlier detection algorithm.

$${z}_{i} = \frac{Xi - \vec{X}}{\overline{S}}$$

Where X̅ and S̅ are the median and MAD. An outlier is called if |^{z}^{ᵢ}|>4. Outliers were excluded from further statistical treatments.

A two-way ANOVA analysis was performed to evaluate the effects of the two factors: age (young, middle-age and old) and gender (M, F) as well as their interaction. P values for both factors and their interaction were computed. Means and standard error for each age group were also computed.

A pair-wise T-test was used to compare the difference between means of the three age groups. Multiple comparisons were adjusted using Hommel's method to control family-wise error. Statistical analysis included natural log (ln) of feline flow cytometry lymphocyte, granulocyte and monocyte data.

Table 1 shows feline peripheral blood leukocyte populations as determined by peripheral blood smear/differential stain (ds, % of total) and flow cytometry (fc). SE represents standard error of the mean and ln represents natural log.

Table 2 shows a two-way ANOVA analysis of age and gender on feline peripheral blood leukocyte populations as determined by peripheral blood smear/differential stain (ds, % of total) and flow cytometry (fc). P values for age, gender and their interaction are indicated as well as for the pair-wise T-test between age groups. Ln represents natural log.

TABLE 1

| | Young Mean ± | Middle Mean ± | Old Mean ± |
|---|---|---|---|
| | SE | SE | SE |
| Neutrophils (ds) | 46.64 ± 4.05 | 54.64 ± 3.41 | 69.33 ± 2.84 |
| Lymphocytes (ds) | 43.91 ± 5.18 | 37.36 ± 3.95 | 23.33 ± 2.72 |
| Eosinophils (ds) | 6.64 ± 1.41 | 5.55 ± 1.32 | 3.75 ± 0.65 |
| CD4 (fc) | 36.54 ± 3.95 | 33.35 ± 2.98 | 38.57 ± 2.63 |
| CD8 (fc) | 6.86 ± 1.27 | 6.25 ± 1.1 | 7.23 ± 1.56 |
| CD4/CD8 (fc) | 6.87 ± 1.37 | 5.66 ± 0.55 | 7.47 ± 1.07 |
| CD4 + CD8 (fc) | 43.4 ± 4.62 | 39.59 ± 3.33 | 45.8 ± 2.93 |
| CD5 (fc) | 66.81 ± 1.66 | 60.74 ± 4.53 | 64.24 ± 2.41 |
| CD45R (fc) | 10.82 ± 1.62 | 12.78 ± 1.61 | 13.3 ± 1.65 |
| Lymphocytes | 274.82 ± 58.44 | 153.09 ± 33.16 | 149.27 ± 26.51 |
| (ln, fc) | | | |
| Granulocytes | 414.73 ± 102.64 | 283.7 ± 71.96 | 253.09 ± 71.16 |
| (ln, fc) | | | |
| Monocytes (ln, fc) | 170.64 ± 27.47 | 177.55 ± 39.22 | 146.91 ± 29.66 |

TABLE 2

| | P Age | P Sex | P Age-Sex | P Yng-Mid | P Yng-Old | P Mid-Old |
|---|---|---|---|---|---|---|
| Neutrophils (ds) | 1.00E−04 | 0.1485 | 0.2328 | 0.1151 | 2.00E−04 | 0.0095 |
| Lymphocytes (ds) | 0.003 | 0.1192 | 0.5523 | 0.2637 | 0.0028 | 0.0365 |
| Eosinophils (ds) | 0.1914 | 0.4759 | 0.1045 | 0.5149 | 0.2544 | 0.5149 |
| CD4 (fc) | 0.5581 | 0.6518 | 0.8609 | 0.6627 | 0.6627 | 0.6627 |
| CD8 (fc) | 0.8822 | 0.6913 | 0.6528 | 0.8494 | 0.8494 | 0.8494 |
| CD4/CD8 (fc) | 0.5463 | 0.993 | 0.8188 | 0.6997 | 0.6997 | 0.672 |
| CD4 + CD8 (fc) | 0.5372 | 0.8018 | 0.9759 | 0.6523 | 0.6523 | 0.6523 |
| CD5 (fc) | 0.4285 | 0.4139 | 0.7367 | 0.5541 | 0.5791 | 0.5791 |
| CD45R (fc) | 0.5293 | 0.1198 | 0.3259 | 0.8145 | 0.6166 | 0.8302 |
| Lymphocytes (ln, fc) | 0.0884 | 0.9666 | 0.7691 | 0.0981 | 0.0856 | 0.9491 |
| Granulocytes (ln, fc) | 0.3865 | 0.8705 | 0.401 | 0.5872 | 0.4404 | 0.8044 |
| Monocytes (ln, fc) | 0.8039 | 0.7551 | 0.735 | 0.883 | 0.883 | 0.883 |

### Example 2

Microarray construction: Lymphocytes were isolated from whole blood and total RNA was extracted. Lymphocytes were also isolated and cultured. These were stimulated with various immunological agents (calcium inophore, 1 ug/ml, 2 hrs; lipopolysaccharide, 20 ng/ml, overnight; IL-2 (rat), 0.2 ng/ml, overnight; IL-4 (feline), 25 ng/ml, overnight; TGF-beta1 (human), 0.05 ng/ml, overnight; IFN-gamma (feline), 1 ng/ml, overnight; PMA+PHA 50 ng/ml each, overnight; no stimulation overnight; ethanol, 2 hrs.). After stimulation, total RNA was extracted and combined with the above RNA. RNA was checked for quality and quantity and shipped to Invitrogen for construction of normalized cDNA libraries. The pCMVSport 6.1 vector was used for cloning in DH10B-Ton A bacteria. Normalization resulted in an 80-fold reduction in beta-actin message with a 96% vector insert rate.

The libraries were plated and approximately 2550 colonies were isolated. Once these were amplified by growth, the associated vectors were isolated and sequenced. Sequencing quality was assessed using phred scores of ≧20. (phred scores are defined as −log(1 error/number of bases) therefore a phred score of 20 is defined as one or fewer errors per 100 bases) This resulted in 89% good quality sequences. cDNA vector inserts were amplified by PCR in 27, 96-well plates. They were then spotted onto prepared microarray slides. The resulting microarrays now represent medium-density lymphocyte gene microarrays of approximately 5100 spots containing 2550 gene targets in duplicate.

Microarray analysis: cDNA was synthesized from 5 ug total RNA according to manufacturer's directions (Genisphere, kit H500130). Briefly, primers constructed with an extension sequence to capture a Cy3 label were incubated with RNA at 80° C. for 10 minutes. Superscript™ II ((Life Technologies, Grand Island, N.Y.) reverse transcriptase was used according to manufacturer's directions. Reverse transcription was performed at 42° C. for 2 hours. Reaction was stopped by the addition of NaOH/EDTA, incubated at 65° C. for 10 minutes and Tris-HCL, pH 7.5 was added to neutralize. cDNA was isolated using Microcon® YM-30 (EMD Millipore Corp., Billerica, Mass.) columns according to manufacturer's directions. Hybridization was carried out with 2.5 ug cDNA, as measured using a NanoDrop (ThermoFisher Scientific Inc.) spectrometer. Microarray hybridization, washes and slide drying procedures were carried out in an automated hybridization system (Tecan Croup Ltd., Männedorf, Switzerland). Briefly, microarrays were hybridized at 38° C. for 18 hours They were washed with 2×SSC, 0.2% SDS (20×SSC; 175.3 g Sodium Chloride and 88.2 g Sodium Citrate per liter, pH 7. 10% SDS; 100 g Sodium Lauryl Sulfate per liter, pH 7.2) @ 42° C., 2×SSC @ 23° C. and 0.2×SSC @ 23° C. The Cy3 label was added to the microarrays and hybed at 23° C. for 3 hours. The previous wash steps were repeated. The microarrays were dried using a Nitrogen gas purge for 2 minutes, 30 seconds.

Transcriptomics. 27 felines from 3 age groups (<7, 7-11 and >11) were used to investigate leukocyte gene expression changes. After 4 arrays were removed due to poor correlation, a total of 23 were used with 5 coming from the young group (<7 years of age), 10 coming from the middle-age group (7-11 years of age) and 8 coming from the old group (>11 years of age).

Gene ID, signal median, background median, and quality control flag information were extracted from the raw data. A gene's expression was determined as the difference between its signal median and its background median. Genes with gene ID as “BLANK”, “Alien”, “n/a”, “blank” or “Blank” were removed. Quality control flagged genes were also eliminated. Within an array, two technical duplicates were combined and their average was calculated. Binary logarithm transformation was used for each gene's expression.

Including the omission of quality controlled flagged spots from the microarray analysis, there was approximately 4% missing data (considering the entire probe-set on the microarray) for the feline analysis. Non-linear cubic spline normalization method was used.

A two-way ANOVA analysis was performed to evaluate the effects of the two factors: age (young, middle-age, old) and gender (M, F) as well as their interaction. P values for both factors and their interaction were computed. A T-test was used to compare the difference between means of the age groups. P values and means of each age group were computed. Each age group should have at least two valid data points in order to enter the comparison with other groups. Feline leukocyte age-related transcriptional changes (p<0.05) are shown in Table 3.

TABLE 3

| | P Value | | | | | | | | |
|---|---|---|---|---|---|---|---|---|---|
| Probe | | Yng | Yng | Mid | Mean Value | | | | |
| ID | Age | Mid | Old | Old | Yng | Mid | Old | Gene Symbol | Gene Description |
| FR15B4 | 0.00 | 0.51 | 0.01 | 0.01 | 10.49 | 10.28 | 11.19 | NA | NA |
| FR5F11 | 0.00 | 0.14 | 0.01 | 0.14 | 8.21 | 8.53 | 8.86 | NA | NA |
| FR14F12 | 0.00 | 0.75 | 0.14 | 0.02 | 9.36 | 9.30 | 9.91 | PPP1R7 | protein phosphatase 1, |
| | | | | | | | | | regulatory (inhibitor) |
| | | | | | | | | | subunit 7 |
| FR8H6 | 0.00 | 0.74 | 0.04 | 0.01 | 15.62 | 15.56 | 15.92 | NA | NA |
| FR7H10 | 0.00 | 0.32 | 0.16 | 0.03 | 9.16 | 9.37 | 9.00 | NA | NA |
| FR19E7 | 0.00 | 0.71 | 0.17 | 0.02 | 10.44 | 10.51 | 10.12 | NA | NA |
| FR2H12 | 0.00 | 0.52 | 0.13 | 0.01 | 14.34 | 14.60 | 13.40 | CFL1 | cofilin 1 (non-muscle) |
| FR18G3 | 0.00 | 0.82 | 0.08 | 0.03 | 9.62 | 9.67 | 10.29 | CCDC45 | coiled-coil domain |
| | | | | | | | | | containing 45 |
| FR27D10 | 0.00 | 0.04 | 0.22 | 0.13 | 14.20 | 14.90 | 14.51 | RBI | retinoblastoma 1 |
| FR17C4 | 0.00 | 0.10 | 0.02 | 0.13 | 15.51 | 15.19 | 14.92 | WIF1 | WNT inhibitory factor 1 |
| FR19E6 | 0.00 | 0.56 | 0.03 | 0.00 | 9.49 | 9.68 | 8.89 | NA | NA |
| FR13F8 | 0.00 | 0.68 | 0.05 | 0.01 | 15.06 | 15.26 | 13.17 | ATP6AP1 | ATPase, H+ |
| | | | | | | | | | transporting, |
| | | | | | | | | | lysosomal accessory |
| | | | | | | | | | protein 1 |
| FR5A9 | 0.00 | 0.64 | 0.08 | 0.01 | 12.38 | 12.18 | 13.26 | CYB5R3 | cytochrome b5 |
| | | | | | | | | | reductase 3 |
| FR19A12 | 0.00 | 0.32 | 0.17 | 0.00 | 8.33 | 8.46 | 8.13 | TREX1 | three prime repair |
| | | | | | | | | | exonuclease 1 |
| FR12A12 | 0.01 | 0.03 | 0.00 | 0.01 | 9.22 | 9.45 | 9.82 | NA | NA |
| FR13E9 | 0.01 | 0.03 | 0.01 | 0.16 | 10.28 | 10.80 | 11.12 | C5AR1 | complement |
| | | | | | | | | | component 5a receptor 1 |
| FR7G9 | 0.01 | 0.16 | 0.05 | 0.21 | 9.30 | 8.99 | 8.82 | TXNDC12 | thioredoxin domain |
| | | | | | | | | | containing 12 |
| | | | | | | | | | (endoplasmic |
| | | | | | | | | | reticulum) |
| FR7A8 | 0.01 | 0.21 | 0.40 | 0.03 | 10.55 | 10.23 | 10.79 | NA | NA |
| FR14A8 | 0.01 | 0.01 | 0.42 | 0.01 | 8.31 | 8.60 | 8.37 | LRRC33 | leucine rich repeat |
| | | | | | | | | | containing 33 |
| FR6F4 | 0.01 | 0.47 | 0.09 | 0.01 | 9.46 | 9.17 | 8.68 | AGRN | agrin |
| FR16G8 | 0.01 | 0.27 | 0.24 | 0.00 | 8.85 | 9.06 | 8.59 | G3BP1 | GTPase activating |
| | | | | | | | | | protein (SH3 domain) |
| | | | | | | | | | binding protein 1 |
| FR24B10 | 0.01 | 0.41 | 0.01 | 0.03 | 9.60 | 9.36 | 8.75 | NA | NA |
| FR25D1 | 0.01 | 0.88 | 0.05 | 0.02 | 10.61 | 10.51 | 9.23 | NA | NA |
| FR5F5 | 0.01 | 0.34 | 0.30 | 0.01 | 12.89 | 12.59 | 13.40 | HSD17B12 | hydroxysteroid (17- |
| | | | | | | | | | beta) dehydrogenase |
| | | | | | | | | | 12 |
| FR5D11 | 0.01 | 0.68 | 0.05 | 0.02 | 12.25 | 12.04 | 11.00 | NA | NA |
| FR1H3 | 0.01 | 0.96 | 0.08 | 0.02 | 9.23 | 9.24 | 9.68 | NA | NA |
| FR26G12 | 0.01 | 0.76 | 0.00 | 0.02 | 9.18 | 9.10 | 8.58 | PITPNC1 | phosphatidylinositol |
| | | | | | | | | | transfer protein, |
| | | | | | | | | | cytoplasmic 1 |
| FR19D2 | 0.01 | 0.86 | 0.00 | 0.01 | 9.26 | 9.32 | 8.57 | NA | NA |
| FR11F12 | 0.01 | 0.75 | 0.01 | 0.00 | 12.46 | 12.36 | 11.54 | NA | NA |
| FR25D4 | 0.01 | 0.51 | 0.13 | 0.01 | 10.12 | 10.39 | 9.52 | NCF1 | neutrophil cytosolic |
| | | | | | | | | | factor 1 |
| FR7G6 | 0.01 | 0.54 | 0.21 | 0.01 | 8.94 | 9.16 | 8.56 | NA | NA |
| FR20A8 | 0.01 | 0.06 | 0.45 | 0.01 | 11.94 | 12.78 | 11.70 | FKBP15 | FK506 binding protein |
| | | | | | | | | | 15, 133 kDa |
| FR12A8 | 0.01 | 0.82 | 0.01 | 0.00 | 13.44 | 13.51 | 14.48 | GGH | gamma-glutamyl |
| | | | | | | | | | hydrolase (conjugase, |
| | | | | | | | | | folylpolygammaglutamyl |
| | | | | | | | | | hydrolase) |
| FR15E12 | 0.02 | 0.03 | 0.20 | 0.13 | 7.81 | 7.22 | 7.48 | CFL1 | cofilin 1 (non-muscle) |
| FR20B10 | 0.02 | 0.06 | 0.59 | 0.01 | 9.47 | 9.82 | 9.37 | MAP3K3 | mitogen-activated |
| | | | | | | | | | protein kinase kinase |
| | | | | | | | | | kinase 3 |
| FR13F1 | 0.02 | 0.85 | 0.02 | 0.00 | 10.63 | 10.59 | 11.21 | ALOX5 | arachidonate 5- |
| | | | | | | | | | lipoxygenase |
| FR10F8 | 0.02 | 1.00 | 0.01 | 0.00 | 9.55 | 9.55 | 10.45 | KIAA1310 | KIAA1310 |
| FR3A1 | 0.02 | 0.70 | 0.05 | 0.02 | 8.69 | 8.81 | 8.13 | DCAF11 | DDB1 and CUL4 |
| | | | | | | | | | associated factor 11 |
| FR22C12 | 0.02 | 0.02 | 0.03 | 0.50 | 8.48 | 8.18 | 8.11 | LY6E | lymphocyte antigen 6 |
| | | | | | | | | | complex, locus E |
| FR24E4 | 0.02 | 0.54 | 0.02 | 0.03 | 11.78 | 11.96 | 12.76 | NUCB1 | nucleobindin 1 |
| FR8C12 | 0.02 | 0.45 | 0.25 | 0.04 | 8.40 | 8.53 | 8.21 | GON4L | gon-4-like (C. elegans) |
| FR11D11 | 0.02 | 0.95 | 0.00 | 0.00 | 10.99 | 10.97 | 11.87 | ELK1 | ELK1, member of |
| | | | | | | | | | ETS oncogene family |
| FR16G6 | 0.02 | 0.14 | 0.26 | 0.00 | 9.86 | 9.58 | 10.15 | SYNGR2 | synaptogyrin 2 |
| FR25G8 | 0.02 | 0.60 | 0.07 | 0.01 | 8.26 | 8.31 | 8.08 | DIAPH1 | diaphanous homolog 1 |
| | | | | | | | | | (Drosophila) |
| FR13B4 | 0.02 | 0.90 | 0.02 | 0.00 | 9.29 | 9.31 | 8.89 | CDC37 | cell division cycle 37 |
| | | | | | | | | | homolog (S. cerevisiae) |
| FR27G6 | 0.02 | 0.29 | 0.05 | 0.03 | 8.87 | 8.60 | 8.30 | MAPK13 | mitogen-activated |
| | | | | | | | | | protein kinase 13 |
| FR12C12 | 0.02 | 0.51 | 0.00 | 0.01 | 11.29 | 11.55 | 12.41 | TRIM50 | tripartite motif- |
| | | | | | | | | | containing 50 |
| FR21D11 | 0.02 | 0.18 | 0.50 | 0.01 | 8.69 | 8.53 | 8.77 | PDLIM7 | PDZ and LIM domain |
| | | | | | | | | | 7 (enigma) |
| FR2C12 | 0.02 | 0.05 | 0.38 | 0.05 | 7.95 | 8.28 | 8.03 | RALY | RNA binding protein, |
| | | | | | | | | | autoantigenic |
| | | | | | | | | | (hnRNP-associated |
| | | | | | | | | | with lethal yellow |
| | | | | | | | | | homolog (mouse)) |
| FR26D12 | 0.02 | 0.70 | 0.03 | 0.01 | 10.99 | 10.84 | 11.66 | CCT7 | chaperonin containing |
| | | | | | | | | | TCP1, subunit 7 (eta) |
| FR4D8 | 0.02 | 0.46 | 0.16 | 0.01 | 7.05 | 7.36 | 6.49 | NA | NA |
| FR6H10 | 0.02 | 0.16 | 0.38 | 0.02 | 6.76 | 6.62 | 6.87 | VWF | von Willebrand factor |
| FR27F12 | 0.02 | 0.12 | 0.01 | 0.22 | 6.22 | 5.96 | 5.79 | NA | NA |
| FR26F5 | 0.02 | 0.18 | 0.59 | 0.03 | 8.33 | 8.53 | 8.28 | NA | NA |
| FR9H7 | 0.03 | 0.47 | 0.12 | 0.04 | 15.91 | 15.84 | 14.49 | NA | NA |
| FR10F4 | 0.03 | 0.05 | 0.00 | 0.00 | 10.04 | 9.63 | 9.04 | FAM32A | family with sequence |
| | | | | | | | | | similarity 32, member A |
| FR14C8 | 0.03 | 0.00 | 0.08 | 0.03 | 4.27 | 5.30 | 4.87 | PDIA6 | protein disulfide |
| | | | | | | | | | isomerase family A, |
| | | | | | | | | | member 6 |
| FR23F1 | 0.03 | 0.42 | 0.20 | 0.02 | 9.98 | 9.82 | 10.39 | PGD | phosphogluconate |
| | | | | | | | | | dehydrogenase |
| FR19C5 | 0.03 | 0.08 | 0.52 | 0.01 | 5.89 | 6.23 | 5.78 | TKT | transketolase |
| FR12B4 | 0.03 | 0.03 | 0.42 | 0.00 | 10.07 | 10.83 | 9.92 | SPOCK2 | sparc/osteonectin, |
| | | | | | | | | | cwcv and kazal-like |
| | | | | | | | | | domains proteoglycan |
| | | | | | | | | | (testican) 2 |
| FR24F2 | 0.03 | 0.91 | 0.06 | 0.02 | 12.37 | 12.33 | 13.15 | C10orf58 | chromosome 10 open |
| | | | | | | | | | reading frame 58 |
| FR5B8 | 0.03 | 0.46 | 0.14 | 0.02 | 8.20 | 8.43 | 7.88 | FLNA | filamin A, alpha |
| FR22B8 | 0.03 | 0.23 | 0.25 | 0.00 | 9.84 | 10.29 | 9.38 | NA | NA |
| FR5E7 | 0.03 | 0.31 | 0.08 | 0.09 | 9.00 | 9.15 | 9.59 | ADNP2 | ADNP homeobox 2 |
| FR26C3 | 0.03 | 0.30 | 0.18 | 0.01 | 7.06 | 6.95 | 7.18 | NA | NA |
| FR16F12 | 0.03 | 0.01 | 0.70 | 0.02 | 10.84 | 11.87 | 10.96 | SIRT6 | sirtuin (silent mating |
| | | | | | | | | | type information |
| | | | | | | | | | regulation 2 homolog) |
| | | | | | | | | | 6 (S. cerevisiae) |
| FR14B12 | 0.03 | 0.54 | 0.03 | 0.00 | 11.44 | 12.20 | 9.21 | NA | NA |
| FR7E3 | 0.03 | 0.02 | 0.11 | 0.28 | 8.71 | 9.20 | 9.01 | NA | NA |
| FR27D11 | 0.03 | 0.37 | 0.16 | 0.01 | 6.69 | 6.87 | 6.48 | NA | NA |
| FR7A4 | 0.03 | 0.17 | 0.04 | 0.11 | 8.88 | 8.56 | 8.30 | NA | NA |
| FR18A7 | 0.03 | 0.84 | 0.07 | 0.02 | 9.06 | 9.11 | 9.70 | HIST1H4I | histone cluster 1, H4i |
| FR3D5 | 0.03 | 0.02 | 0.31 | 0.05 | 12.10 | 11.35 | 11.80 | ILK | integrin-linked kinase |
| FR1H1 | 0.03 | 0.54 | 0.11 | 0.02 | 11.83 | 11.56 | 12.58 | PHC2 | polyhomeotic |
| | | | | | | | | | homolog 2 |
| | | | | | | | | | (Drosophila) |
| FR6A4 | 0.04 | 0.85 | 0.08 | 0.02 | 10.17 | 10.14 | 10.53 | LOC100128934 | similar to FXYD |
| | | | | | | | | | domain-containing ion |
| | | | | | | | | | transport regulator 6 |
| FR15D4 | 0.04 | 0.82 | 0.00 | 0.00 | 9.98 | 9.89 | 10.94 | NA | NA |
| FR3B8 | 0.04 | 0.76 | 0.11 | 0.02 | 11.66 | 11.77 | 10.55 | DNAJA2 | DnaJ (Hsp40) |
| | | | | | | | | | homolog, subfamily |
| | | | | | | | | | A, member 2 |
| FR8A4 | 0.04 | 0.05 | 0.79 | 0.02 | 9.62 | 9.14 | 9.55 | NA | NA |
| FR11B4 | 0.04 | 0.39 | 0.00 | 0.01 | 10.61 | 10.93 | 11.94 | WTAP | Wilms tumor 1 |
| | | | | | | | | | associated protein |
| FR25C4 | 0.04 | 0.09 | 0.70 | 0.00 | 10.27 | 10.72 | 10.16 | NA | NA |
| FR16C6 | 0.04 | 0.04 | 0.82 | 0.01 | 8.52 | 8.85 | 8.50 | SH3BP1 | SH3-domain binding |
| | | | | | | | | | protein 1 |
| FR3B9 | 0.04 | 0.09 | 0.04 | 0.27 | 11.19 | 11.87 | 12.23 | NA | NA |
| FR7A5 | 0.04 | 0.11 | 0.41 | 0.02 | 10.10 | 10.76 | 9.53 | NA | NA |
| FR9H1 | 0.04 | 0.82 | 0.02 | 0.03 | 9.97 | 10.04 | 10.57 | NA | NA |
| FR16B4 | 0.04 | 0.37 | 0.01 | 0.01 | 12.93 | 12.54 | 11.39 | NA | NA |
| FR21G6 | 0.04 | 0.49 | 0.03 | 0.04 | 10.99 | 10.78 | 9.99 | NA | NA |
| FR16E6 | 0.04 | 0.51 | 0.02 | 0.01 | 8.99 | 8.89 | 8.39 | ABHD5 | abhydrolase domain |
| | | | | | | | | | containing 5 |
| FR21H6 | 0.04 | 0.70 | 0.09 | 0.01 | 15.86 | 15.89 | 14.49 | NA | NA |
| FR26H5 | 0.04 | 0.10 | 0.00 | 0.18 | 10.69 | 10.38 | 10.16 | NA | NA |
| FR13D8 | 0.04 | 0.93 | 0.03 | 0.01 | 9.52 | 9.49 | 8.93 | CHMP2A | chromatin modifying |
| | | | | | | | | | protein 2A |
| FR26G5 | 0.04 | 0.89 | 0.04 | 0.00 | 10.42 | 10.49 | 9.58 | NA | NA |
| FR23C1 | 0.04 | 0.77 | 0.02 | 0.02 | 10.33 | 10.25 | 9.55 | ST6GALNAC4 | ST6 (alpha-N-acetyl- |
| | | | | | | | | | neuraminyl-2,3-beta- |
| | | | | | | | | | galactosyl-1,3)-N- |
| | | | | | | | | | acetylgalactosaminide |
| | | | | | | | | | alpha-2,6- |
| | | | | | | | | | sialyltransferase 4 |
| FR5A4 | 0.04 | 0.68 | 0.10 | 0.02 | 14.08 | 14.43 | 12.06 | NA | NA |
| FR6B4 | 0.04 | 0.21 | 0.05 | 0.15 | 9.09 | 8.89 | 8.67 | NA | NA |
| FR11C6 | 0.05 | 0.44 | 0.00 | 0.00 | 10.97 | 11.15 | 11.90 | NA | NA |
| FR17H11 | 0.05 | 0.46 | 0.06 | 0.02 | 14.08 | 13.90 | 13.40 | NA | NA |
| FR21C8 | 0.05 | 0.71 | 0.04 | 0.02 | 11.24 | 11.16 | 10.73 | NA | NA |
| FR26H10 | 0.05 | 0.28 | 0.32 | 0.04 | 9.62 | 9.46 | 9.94 | NA | NA |
| FR10B7 | 0.05 | 0.30 | 0.01 | 0.02 | 9.85 | 9.56 | 9.02 | FKBP8 | FK506 binding protein |
| | | | | | | | | | 8, 38 kDa |
| FR14B4 | 0.05 | 0.49 | 0.04 | 0.00 | 9.01 | 8.88 | 8.53 | NA | NA |
| FR21F9 | 0.05 | 0.33 | 0.01 | 0.04 | 10.89 | 10.49 | 9.63 | NHP2L1 | NHP2 non-histone |
| | | | | | | | | | chromosome protein |
| | | | | | | | | | 2-like 1 (S. cerevisiae) |
| FR12D12 | 0.05 | 0.04 | 0.00 | 0.14 | 9.93 | 10.50 | 10.79 | SLC25A29 | solute carrier family |
| | | | | | | | | | 25, member 29 |
| FR21B12 | 0.05 | 0.17 | 0.20 | 0.01 | 10.54 | 10.15 | 10.97 | TALDO1 | transaldolase 1 |
| FR22C11 | 0.05 | 0.67 | 0.09 | 0.00 | 14.56 | 14.77 | 13.50 | ACTL6B | actin-like 6B |
| FR12F12 | 0.05 | 0.20 | 0.06 | 0.00 | 7.97 | 8.55 | 7.09 | ZC3H7A | zinc finger CCCH- |
| | | | | | | | | | type containing 7A |

### Example 3

Protein Analysis. Cytokine and endocrine protein levels were determined using the feline Milliplex kit (Millipore, St. Charles, Mo.) according to manufacturer's directions. Specifically, 200 μl of Wash buffer was added per well and shaken 10 minutes at room temp. This was vacuumed out and 25 μl standards, controls and background (assay buffer) was added to appropriate wells. 25 μls of serum matrix was added to the standards, controls and background (assay buffer) was added to appropriate wells. 25 μl of plasma was added to the sample wells followed by 25 μl of beads. This was incubated overnight on a shaking plate at 4° C. Fluid was removed gently by vacuum and the plates washed 2 times with 200 μls of wash buffer. 25 μl of detection antibody was added and incubated with shaking for 1 hour at room temperature. 25 μl streptavidin-Phycoerythrin was added and incubated 30 minutes with shaking. Fluid was removed gently by vacuum and washed 3 times. 100 μl sheath fluid was added and the beads resuspended on a shaker plate for 5 minutes. The plate was then run on the Luminex 100 IS according to manufacturer's directions. Samples were run in duplicate.

36 samples (initially n=12) were run for young (<7 years of age), middle-aged (7-11 years of age) and old (>11 years of age). Final n values were based on successful determination of protein levels (Table 5). P values were determined using a T-test to compare the difference between means of the three age groups (Table 4). Means and standard errors were calculated. Results are shown in Table 4.

TABLE 4

| | P | | | | | | | | |
|---|---|---|---|---|---|---|---|---|---|
| | | Old- | Old- | Mean | | | SE | | |
| | Mid-Yng | Mid | Yng | Yng | Mid | Old | Yng | Mid | Old |
| Leptin | 0.797 | 0.027 | 0.003 | 1068.9 | 984.4 | 308.6 | 194.2 | 258.9 | 64.1 |
| MCP-1 | 0.098 | 0.797 | 0.070 | 483.3 | 249.5 | 225.1 | 79.9 | 73.5 | 46.2 |
| IFN | 0.178 | 0.335 | 0.104 | 2121.8 | 751.9 | 440.0 | 875.6 | 273.3 | 137.3 |
| IL- | 0.146 | 0.423 | 0.107 | 2237.5 | 574.8 | 367.5 | 984.4 | 227.0 | 102.0 |
| 12p40 | | | | | | | | | |
| SCF | 0.207 | 0.331 | 0.111 | 791.4 | 445.0 | 285.1 | 184.9 | 137.9 | 10.1 |
| IL-18 | 0.073 | 0.909 | 0.115 | 1729.1 | 678.2 | 715.8 | 517.5 | 48.5 | 310.6 |
| SDF-1 | 0.558 | 0.298 | 0.147 | 1773.8 | 2865.9 | 310.2 | 850.0 | 1307.8 | 143.3 |
| GLP-1 | 0.155 | 0.895 | 0.165 | 140.8 | 30.7 | 32.9 | 68.7 | 9.1 | 13.6 |
| IL-4 | 0.335 | 0.448 | 0.171 | 1517.7 | 688.8 | 378.9 | 728.6 | 356.4 | 120.4 |
| PDGF- | NA | NA | 0.277 | 1350.0 | NA | 2156.9 | 253.1 | NA | 432.3 |
| BB | | | | | | | | | |
| Insulin | 0.102 | 0.576 | 0.334 | 193.2 | 129.5 | 155.0 | 20.2 | 30.7 | 32.4 |
| Amylin | NA | NA | 0.529 | 236.4 | 81.4 | 131.7 | 140.8 | NA | 67.7 |
| Flt-3L | 0.239 | NA | NA | 199.8 | 111.0 | 96.0 | 53.9 | 7.7 | NA |
| IL-1b | 0.198 | NA | NA | 377.3 | 198.1 | 142.2 | 108.8 | 21.1 | NA |
| IL-2 | 0.709 | NA | NA | 476.9 | 370.2 | 163.6 | 266.0 | 17.2 | NA |
| IL-6 | 0.296 | NA | NA | 1299.3 | 736.9 | NA | 442.3 | 111.0 | NA |
| IL-10 | 0.651 | NA | NA | 783.2 | 615.9 | 122.6 | 333.2 | 25.6 | NA |

TABLE 5

| | N | | | |
|---|---|---|---|---|
| | Yng | Mid | Old | Units |
| Leptin | 10 | 11 | 12 | pg/mL |
| MCP-1 | 3 | 3 | 2 | pg/mL |
| IFN | 7 | 7 | 7 | pg/mL |
| IL-12p40 | 7 | 9 | 9 | pg/mL |
| SCF | 3 | 4 | 3 | pg/mL |
| IL-18 | 10 | 6 | 7 | pg/mL |
| SDF-1 | 6 | 2 | 2 | pg/mL |
| GLP-1 | 8 | 5 | 5 | pg/mL |
| IL-4 | 7 | 5 | 6 | pg/mL |
| PDGF-BB | 2 | 0 | 2 | pg/mL |
| Insulin | 10 | 10 | 9 | pg/mL |
| Amylin | 5 | 1 | 4 | pg/mL |
| Flt-3L | 3 | 4 | 1 | pg/mL |
| IL-1b | 4 | 2 | 1 | pg/mL |
| IL-2 | 5 | 2 | 1 | pg/mL |
| IL-6 | 4 | 3 | 0 | pg/mL |
| IL-10 | 4 | 2 | 1 | pg/mL |

The specification has disclosed typical preferred embodiments of the invention. Although specific terms are employed, they are used in a generic and descriptive sense only and not for purposes of limitation, the scope of the invention being set forth in the claims. Clearly, many modifications and variations of the invention are possible in light of the above teachings. It is therefore to be understood that within the scope of the appended claims the invention may be practiced otherwise than as specifically described.

What is claimed is:

1. A combination comprising a plurality of biomarkers associated with immune function that are differentially expressed in samples from old animals compared with samples from young animals, wherein the biomarker associated with immune function is a gene expression marker that is one or more of WTAP, NHP2L1, TRIM50, GGH, FAM32A, NUCB1, KIAA1310, ELK1, SLC25A29, C5AR1, FKBP8, ST6GALNAC4, CCT7, PITPNC1, ABHD5, WIF1, ALOX5, CHMP2A, CDC37, and LY6E.

2. The combination of claim 1 wherein the gene expression marker is one or more of WTAP, NHP2L1, TRIM50, GGH, FAM32A, NUCB1, KIAA1310, ELK1, SLC25A29, C5AR1, FKBP8, ST6GALNAC4, CCT7, PITPNC1, ABHD5, WIF1, ALOX5, and CHMP2A.

3. A combination comprising a plurality of biomarkers associated with immune function that are differentially expressed in samples from middle-aged animals compared with samples from young animals, wherein the biomarker associated with immune function is a gene expression marker that is one or more of SIRT6, PDIA6, SPOCK2, ILK, RB1, CFL1, SLC25A29, C5AR1, SH3BP1, LRRC33, and LY6E.

4. The combination of claim 3 wherein the gene expression marker is one or more of SIRT6, PDIA6, SPOCK2, ILK, RB1, and CFL1.

5. A combination comprising a plurality of biomarkers associated with immune function that are differentially expressed in samples from old animals compared with samples from Middle-aged animals, wherein the biomarker associated with immune function is a gene expression marker that is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, HIST1H4I, SYNGR2, PGD, CHMP2A, FLNA, FKBP8, PITPNC1, ABHD5, AGRN, G3BP1, TKT, MAP3K3, CDC37, PDIA6, LOC100128934, SH3BP1, TREX1, GON4L, MAPK13, PDLIM7, LRRC33, VWF, and DIAPH1.

6. The combination of claim 5 wherein the gene expression marker is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, and HIST1H4I.

7. A combination comprising a plurality of biomarkers associated with immune function that are differentially expressed in samples from old animals compared with samples from young animals, wherein the biomarker associated with immune function is a protein that is one or more of Leptin, MCP-1 (monocyte chemotactic protein-1), IFN (Interferon), IL-12p40 (Interleukin-12 subunit beta), SCF (stem cell factor), IL-18 (Interleukin-18), SDF-1 (stromal cell-derived factor-1), GLP-1 (Glucagon-like peptide-1), IL-4 (Interleukin-4), PDGF-BB (Platelet-derived growth factor), Insulin, Amylin, Flt-3L (Fms-like Tyrosine Kinase-3), IL-1b (Interleukin-1 beta), IL-2 (Interleukin-2), IL-6 (Interleukin-6), and IL-10 (Interleukin-10).

8. The combination of claim 7 wherein the protein is one or more of Leptin, MCP-1, IFN, IL-12p40, SCF, IL-18, SDF-1, GLP-1, and IL-4.

9. A combination comprising a plurality of biomarkers associated with immune function that are differentially expressed in samples from old animals compared with samples from middle-aged animals, wherein the biomarker associated with immune function is a protein that is one or more of Leptin, SDF-1, SCF, IFN, IL-12p40, IL-4, Insulin, MCP-1, GLP-1, IL-18, PDGF-BB, Amylin, Flt-3L, IL-1b, IL-2, IL-6, and IL-10.

10. The combination of claim 9 wherein the protein is one or more of Leptin, SDF-1, SCF, and IFN.

11. A combination comprising a plurality of biomarkers associated with immune function that are differentially expressed in samples from middle-aged animals compared with samples from young animals, wherein the biomarker associated with immune function is a protein that is one or more of IL-18, MCP-1, Insulin, IL-12p40, GLP-1, IFN, IL-1b, SCF, Flt-3L, IL-6, IL-4, SDF-1, IL-10, IL-2, Leptin, PDGF-BB, and Amylin.

12. The combination of claim 11 wherein the protein is one or more of IL-18, MCP-1, Insulin, IL-12p40, GLP-1, IFN, and IL-1b.

13. A method for determining if a composition is effective in strengthening the immune function in an animal comprising:
a. obtaining a baseline sample from the animal prior to administration of the composition;
b. analyzing the baseline sample for one or more biomarkers associated with immune function;
c. administering the composition to the animal for a suitable amount of time;
d. obtaining a treatment sample from the animal after completion of the suitable amount of time;
e. analyzing the treatment sample for one or more biomarkers associated with immune function; and
f. determining if the composition is effective if one or more biomarkers present in the baseline sample is differentially expressed in the treatment sample.

14. The method of claim 13 wherein determining if the composition is effective if two or more biomarkers present in the baseline sample are differentially expressed in the treatment sample.

15. The method of claim 13 wherein determining if the composition is effective if three or more biomarkers present in the baseline sample are differentially expressed in the treatment sample.

16. The method of claim 13 wherein the biomarker associated with immune function is a gene expression marker that is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, HIST1H4I, SYNGR2, PGD, CHMP2A, FLNA, FKBP8, PITPNC1, ABHD5, AGRN, G3BP1, TKT, MAP3K3, CDC37, PDIA6, LOC100128934, SH3BP1, TREX1, GON4L, MAPK13, PDLIM7, LRRC33, VWF, DIAPH1, SLC25A29, C5AR1, WIF1, LY6E, ILK, and RB1.

17. The method of claim 13 wherein the biomarker associated with immune function is a gene expression marker that is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, HIST1H4I, SLC25A29, C5AR1, FKBP8, PITPNC1, ABHD5, WIF1, CHMP2A, PDIA6, ILK, and RB1.

18. The method of claim 13 wherein the biomarker associated with immune function is a gene expression marker that is one or more of NHP2L1, TRIM50, GGH, FAM32A, SIRT6 and PDIA6, ATP6AP1, ZC3H7A, ACTL6B, DNAJA2, CFL1, CYB5R3, FKBP15, PHC2, and WTAP.

19. The method of claim 13 wherein the biomarker associated with immune function is a protein that is one or more of Leptin, MCP-1, IFN, IL-12p40, SCF, IL-18, SDF-1, GLP-1, IL-4, PDGF-BB, Insulin, Amylin, Flt-3L, IL-1b, IL-2, IL-6, and IL-10.

20. A method for determining if an animal is responding to treatment with a composition suitable for strengthening immune function comprising:
a. obtaining a baseline sample from the animal prior to administration of the composition;
b. analyzing the baseline sample for one or more biomarkers associated with immune function;
c. administering the composition to the animal for a suitable amount of time;
d. obtaining a treatment sample from the animal after completion of the suitable amount of time;
e. analyzing the treatment sample for one or more biomarkers associated with immune function; and
f. determining if the animal is responding to treatment if one or more biomarker present in the baseline sample is differentially expressed in the treatment sample.

21. The method of claim 20 wherein determining if the animal is responding to treatment if two or more biomarkers present in the baseline sample are differentially expressed in the treatment sample.

22. The method of claim 20 wherein determining if the animal is responding to treatment if three or more biomarkers present in the baseline sample are differentially expressed in the treatment sample.

23. The method of claim 20 wherein the biomarker associated with immune function is a gene expression marker that is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, HIST1H4I, SYNGR2, PGD, CHMP2A, FLNA, FKBP8, PITPNC1, ABHD5, AGRN, G3BP1, TKT, MAP3K3, CDC37, PDIA6, LOC100128934, SH3BP1, TREX1, GON4L, MAPK13, PDLIM7, LRRC33, VWF, DIAPH1, SLC25A29, C5AR1, WIF1, LY6E, ILK, and RB1.

24. The method of claim 20 wherein the biomarker associated with immune function is a gene expression marker that is one or more of ATP6AP1, ZC3H7A, ACTL6B, DNAJA2, CFL1, CYB5R3, FKBP15, PHC2, WTAP, GGH, SPOCK2, SIRT6, ELK1, KIAA1310, NCF1, TRIM50, NHP2L1, CCT7, TALDO1, C10orf58, HSD17B12, NUCB1, ST6GALNAC4, DCAF11, CCDC45, ALOX5, PPP1R7, FAM32A, HIST1H4I, SLC25A29, C5AR1, FKBP8, PITPNC1, ABHD5, WIF1, CHMP2A, PDIA6, ILK, and RB1.

25. The method of claim 20 wherein the biomarker associated with immune function is a gene expression marker that is one or more of NHP2L1, TRIM50, GGH, FAM32A, SIRT6 and PDIA6, ATP6AP1, ZC3H7A, ACTL6B, DNAJA2, CFL1, CYB5R3, FKBP15, PHC2, and WTAP.

26. The method of claim 20 wherein the biomarker associated with immune function is a protein that is one or more of Leptin, MCP-1, IFN, IL-12p40, SCF, IL-18, SDF-1, GLP-1, IL-4, PDGF-BB, Insulin, Amylin, Flt-3L, IL-1b, IL-2, IL-6, and IL-10.

27. The method of claim 20 wherein the biomarker associated with immune function is a protein that is one or more of Leptin and MCP-1.
